(19) United States
(12) Patent Application Publication
(10) Pub. No.: US 2010/0268258 A1
(43) Pub. Date: Oct. 21, 2010

(54) SAFETY SCALPEL

(76) Inventor: Timothy J. Maxwell, Stittsville (CA)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/747,633

(22) PCT Filed: Dec. 11, 2008
(86) PCT No.: PCT/IB2008/003459

§ 371 Date: Jun. 11, 2010

Related U.S. Application Data
(60) Provisional application No. 61/007,329, filed on Dec. 11, 2007.

(51) Int. Cl. A61B 17/3213

(52) U.S. Cl. 606/167

(57) ABSTRACT

A scalpel enabling both safe blade engagement or disengagement and safe passing among personnel during surgical procedures. The scalpel is positionable in three configurations, including a blade shielded position, a blade exposed and operational position and a fully extended position for engagement and disengagement of the blade.

### FIELD OF THE INVENTION

This invention relates to surgical scalpels and, in particular, to surgical scalpels that have disposable, retractable and ejectable blades.

### BACKGROUND

Surgical scalpels are indispensable in the medical field. Numerous types of scalpels are available on the market. Desirable features in a surgical scalpel include safety, convenience of use, and cost. Surgery involves exposure to sharp objects, bodily fluids, and biomedical waste, and can be dangerous for a patient, the surgeon, and the surgery team. It is therefore desirable to have a scalpel that meets stringent occupational health and safety requirements. A scalpel should be designed for safe surgical contact with a patient. It is desirable that a scalpel be capable of being sterilized. It is also desirable that a scalpel blade be retractable and ejectable so that a user does not risk physical contact with the blade. An exposed blade can easily cut or prick someone and bring that person into contact with possibly contaminated or unsanitary material. Because a surgeon may not always have both hands available to handle a scalpel, it is also desirable that a retractable and ejectable scalpel be operable with one hand. Finally, it is desirable that a scalpel not need added lubricant.

### SUMMARY

This invention provides a retractable (or shieldable) and ejectable, disposable or replaceable-blade scalpel assembly that can be easily manipulated with one hand. A blade shield may be slid forward to cover the blade during periods of non-use or slid back to expose the blade for use. The movement can be accomplished with one hand with thumb pressure in the direction of the desired movement. It is common for surgical instruments to be passed between surgical personnel, and because the blade of this scalpel can be covered, the scalpel can be passed from one person to another without risk of contact with the blade. The scalpel's blade is also ejectable by use of thumb pressure in a forward-then-back pattern, to uncover the base of the blade, enabling the blade to be dropped from its position on the handle assembly into a proper receptacle.

Embodiments of the scalpels of this invention permit the significant majority of all disposable scalpel blades to be engaged, concealed, and ejected without any physical contact between the user and the blade.

Among other desirable features of some embodiments of this invention, movement of the blade shield between “blade exposed” and “blade covered” positions is accomplished with intuitively logical pressure on the button in the direction the shield must slide to cover or expose the blade as desired. By contrast, movement of the shield far enough distally on the handle to completely expose the blade for removal and replacement requires a slightly more complex and less intuitive application of force first to the button proximally until the shield moves a small distance proximally on the handle, further covering the blade, and then pressure must be applied distally to the shield (but not to the button) to slide the shield distally until the blade is released. Similarly, movement of the shield beyond the “safe passing,” “blade covered” position on the handle to entirely remove the shield and disassemble the scalpel requires a likewise somewhat more complex and less intuitive application of force. Force must be applied first to the button distally until the shield moves a small distance distally on the handle in the direction opposite that desired, and then pressure must be applied proximally to the shield (but not to the button) to slide the shield proximally until it disengages from the handle. While these sequences of application of pressure to replace the blade or disassemble the scalpel may be easily learned, use of each sequence tends to require intentional action, reducing the likelihood that blade disengagement or scalpel disassembly will be accomplished accidentally.

The scalpels of this invention may be made, among other materials, of stainless steel and high-temperature resistant plastic. Use of these materials permit scalpel sterilization using both steam and chemical sterilization techniques, which protects patients while allowing the scalpel assembly to be reused, in contrast to disposable sterile scalpels that can only be used once.

Scalpels of this invention are compatible with cleaning and sterilization protocol.

Scalpels of this invention are ergonomic to help surgeons maintain proper control of the scalpel during use, and their geometry can provide excellent visualization around the proximal end of the scalpel. The construction of the scalpel is relatively light weight but has sufficient heft to feel familiar and appropriate to experienced surgeons. When engaged, the blade is locked firmly in place and facilitates firm incisions using the scalpel. Raised areas on the assembly components facilitate use and manipulation of the scalpel and its components without slipping.

Scalpels of this invention also function without any need for lubricant, making them convenient and easy to maintain and use.

The distal end of the scalpel of this invention is blunt to serve as a dissector or probe or aide to push tissue out of the way during surgery.

These and numerous other features and benefits of the scalpels of this invention will become apparent by reference to the following descriptions and the accompanying drawings.

### BRIEF DESCRIPTION OF THE DRAWINGS

FIG. 1 is a perspective view of a disposable blade and scalpel assembly according to one embodiment of this invention, shown with the blade exposed.

FIG. 2 is an exploded perspective view of the scalpel assembly shown in FIG. 1.

FIG. 3 is a top view of the handle of the scalpel assembly shown in FIG. 1.

FIG. 4 is a bottom view of the handle shown in FIG. 3.

FIG. 5 is a perspective view of the bottom of the scalpel assembly shown in FIG. 1.

FIG. 6 is a top view of the locking hold-down shown in FIG. 2.

FIG. 7 is a bottom view of the locking hold-down shown in FIG. 6.

FIG. 8 is a profile view of the locking hold-down shown in FIG. 6.

FIG. 9 is a perspective view of the shield shown in FIG. 1.

FIG. 10 is a top view of the shield shown in FIG. 9.

FIG. 11 is a bottom view of the shield shown in FIG. 9.

FIG. 12 is a perspective view of the blade of the scalpel assembly shown in FIG. 1.

FIG. 13 is a perspective view of the locking hold-down shown in FIG. 6.

FIG. 14 is a profile view of the scalpel assembly shown in FIG. 1 with the blade covered.

FIG. 15 is a profile view of the scalpel assembly shown in FIG. 1 with the blade exposed.

FIG. 16 is a profile view of the scalpel assembly shown in FIG. 1 with the blade cover retracted so that the blade is disengaged.

FIG. 17 is a bottom view of the scalpel assembly shown in FIG. 1 with the blade exposed.

FIG. 18 is a top view of the scalpel assembly shown in FIG. 1 with the blade covered.

FIG. 19 is a top view of the scalpel assembly of FIG. 1 with the blade cover retracted so that the blade is disengaged.

FIG. 20 is a bottom view of the scalpel assembly of FIG. 1 with the blade covered.

FIG. 21 is another bottom view of the scalpel assembly of FIG. 1 with the blade exposed.

FIG. 22 is a view, from the proximal end, of the handle shown in FIG. 3.

FIG. 23 is a view, from the proximal end, of the shield shown in FIG. 9.

FIG. 24 is a view, from the distal end, of the shield shown in FIG. 9.

FIG. 25 is a profile view of the button shown in FIG. 2.

FIG. 26 is a perspective view of the button shown in FIG. 2.

FIG. 27 is an enlarged partial perspective view of the shield of FIGS. 9 and 10.

FIG. 28 is an enlarged partial view of the shield and button of FIG. 2, with the button shown in cross section.

FIGS. 29-32 illustrate various views of an alternative embodiment of a scalpel of this invention.

### DETAILED DESCRIPTION

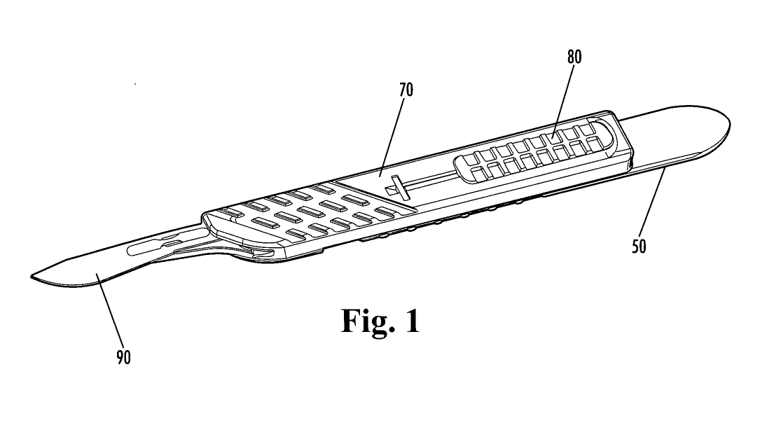

An exemplary scalpel assembly of this invention (illustrated in FIG. 1) includes a handle 50, a hold-down 60 (not shown), a shield 70, and a button 80 and may be assembled with a standard scalpel blade 90.

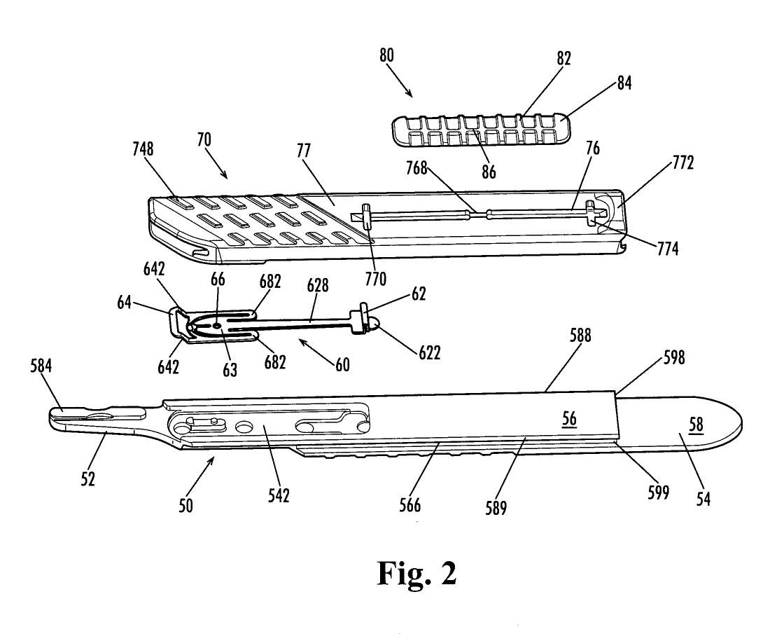

As shown in FIGS. 2-4, the handle 50 may be an elongated bar that may be generally the same size as typical conventional scalpels with a proximal end 52 carrying a blade tang 584 and a blunt, distal or dissector end 54. The hold-down 60 is seated within a hold-down recess 542 in the handle 50, and the shield 70 partially surrounds and slides along the handle 50, with travel limited by the function of the hold-down 60, which is controlled by the button 80 sliding within a longitudinal slot or travel-way 76 in the shield.

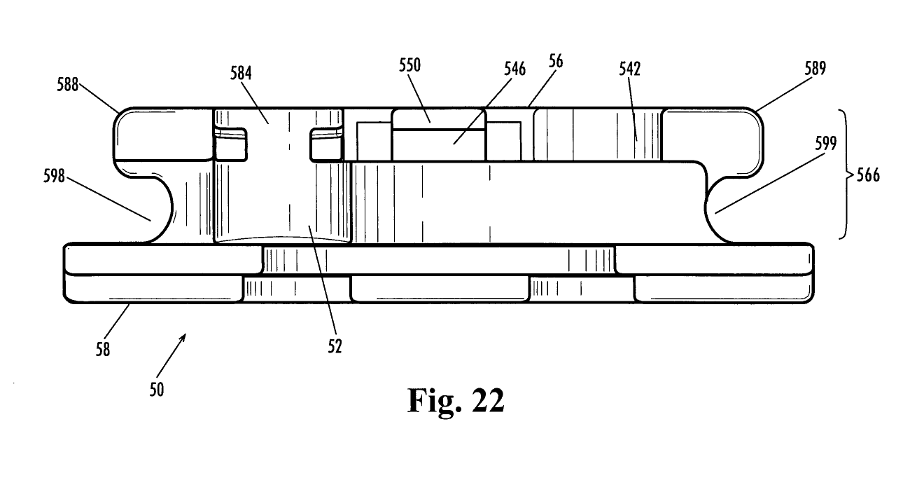

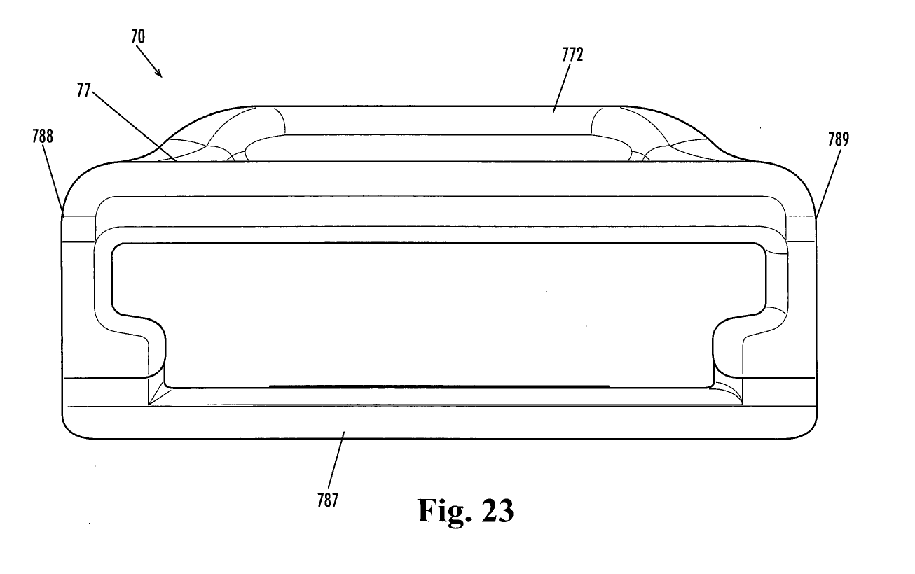

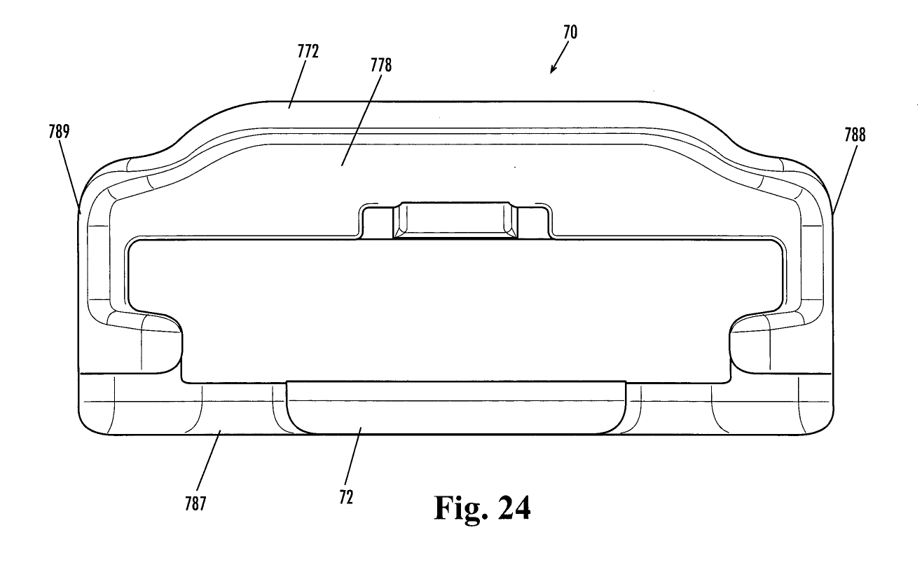

As will be appreciated by reference to FIGS. 2 and 22, handle 50 has a relatively thick middle portion 566 extending from the distal, dissector end 54 to the tang-carrying proximal end 52 to which the blade-receiving tang 584 is attached. As can be particularly well seen in FIG. 22 the face 56 overlies the middle 566 of handle 50 at both the top 588 and bottom 589 of the handle, and the back 58 similarly extends beyond the middle 566 so that opposed grooves 598 and 599 are formed on the top 588 and bottom 589 of the handle 50 between face 56 and back 58.

Shield 70 generally lies against handle face 56 and has top and bottom edges 788 and 789 that extend around the top 588 and bottom 589 of handle face 56 and into the grooves 598 and 599, thereby enabling shield 70 to be assembled onto handle 50 and, as described below, slide on handle 50 among “blade shielded,” “blade exposed” and “blade removal/replacement” positions.

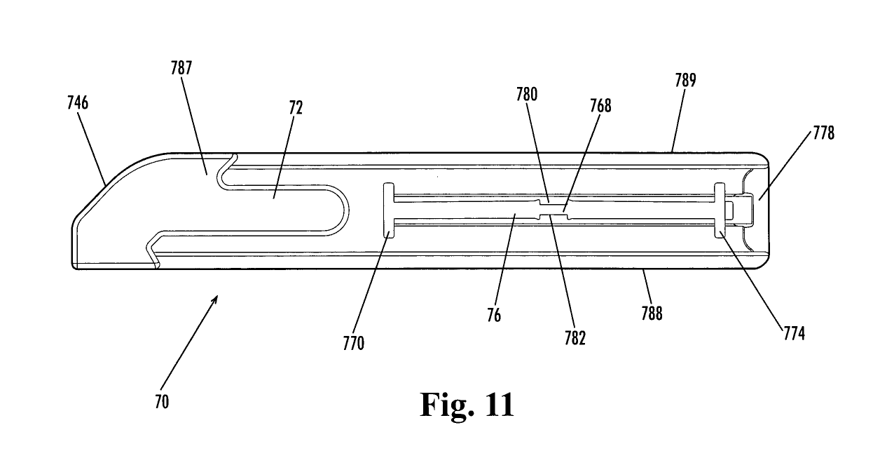

As is shown in FIG. 11, shield 70 edges 788 and 789 are joined by a shield bridge region 787 from which a shield tongue 72 extends distally so that the proximal end of shield 70 provides a sheath substantially surrounding blade 90 when shield 70 is in the “safety” or “blade shielded” position.

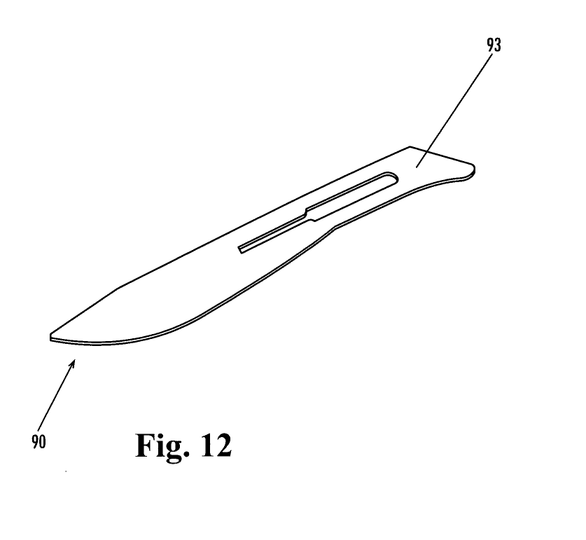

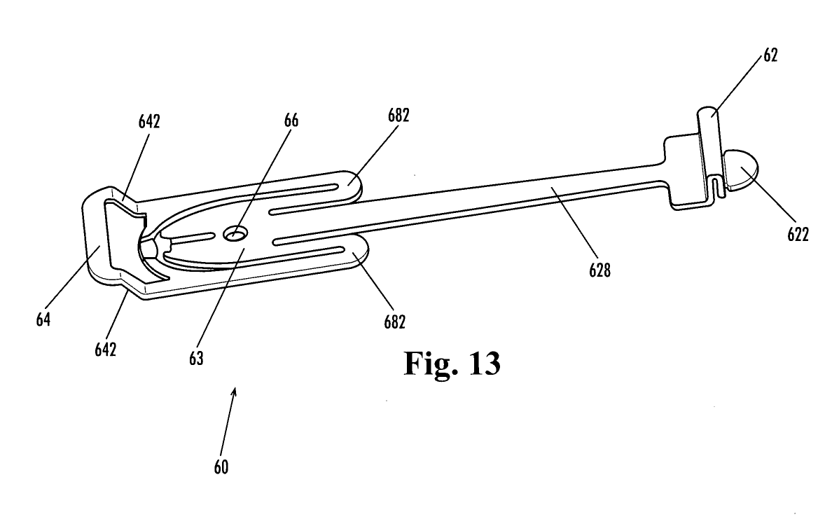

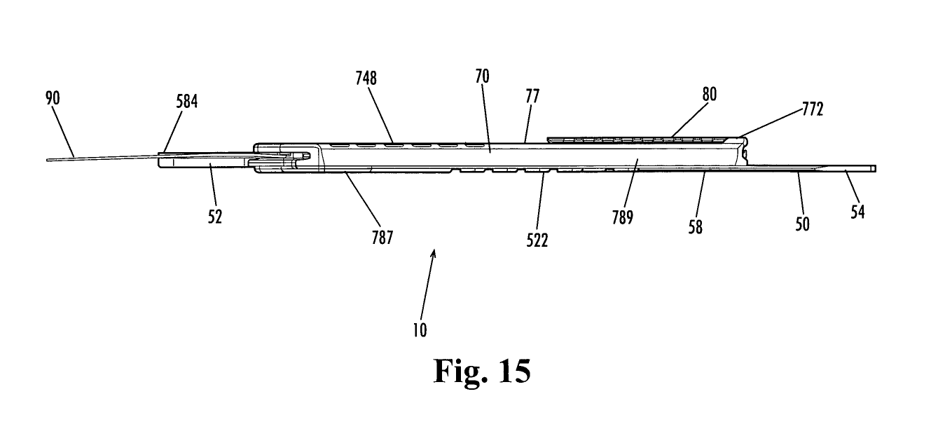

As shown in FIGS. 2, 3, 15 and 16, blade-receiving tang 584 is configured to receive conventional disposable scalpel blades 90 and may have the same structure as the tang described in published Scalpel Assembly patent application PCT/IB00/00426, WO 00/61014, which is incorporated herein by reference. When a disposable blade 90 (shown in FIGS. 12, 15, 16 and 19 for example) is positioned on tang 584 and the distal end 93 of blade 90 is forced or retained against face 65, the blade 90 will be secured to the tang 584 and cannot be removed by any of the forces or movements that occur during normal surgical use of the scalpel assembly 10 (not referenced on figure). Removal of the force or structure retaining the distal end 93 against face 65 allows blade 90 to be disengaged from tang 584 and removed or dropped off of handle 50.

Tang 584 may alternatively have any other structure that will receive and securely hold conventional replaceable surgical blades by application of force to the distal blade end 93 normal to one of the blade sides or otherwise preventing movement of distal blade end 93 so that blade 90 will not move relative to or detach from tang 584 or other structure for receiving and holding blade 90. Such alternative tangs typically have a protrusion that will pass through the broader, distal end of the slot in conventional blades, with a narrower waist or base supporting the protrusion so that the blade can be moved distally until the protrusion is positioned above the narrower, proximal end of the blade slot.

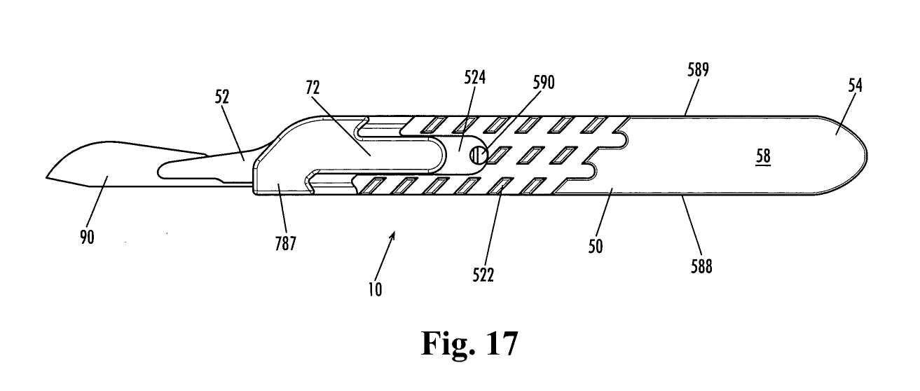

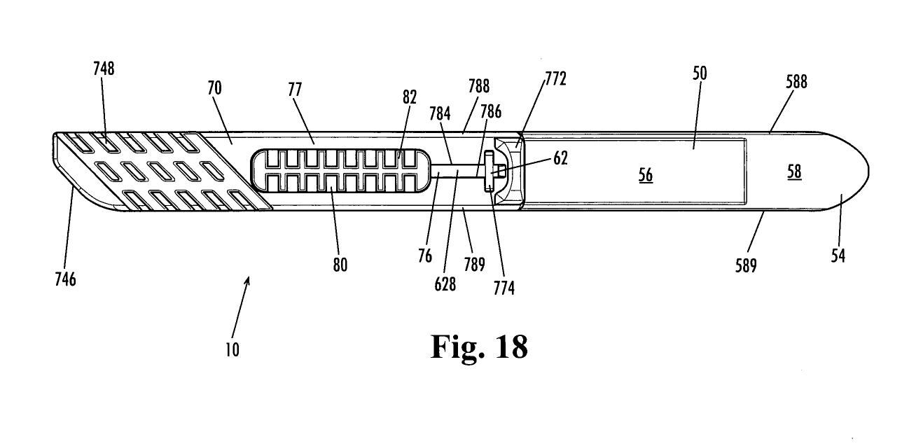

Hold-down 60 cooperates with the other components of handle 50 to retain blade 90 on tang 584 and lock shield 70 in either a blade-covered position (as illustrated in FIG. 18) or a blade-exposed position (as illustrated in FIG. 17).

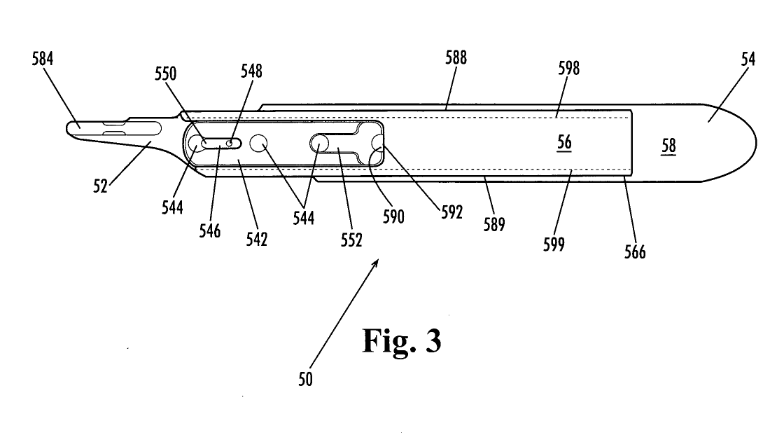

Hold-down 60 is positioned within a hold-down recess 542 in handle 50, shown in FIGS. 2 and 3. Recess 542 is an elongated depression of sufficient depth to receive and retain hold-down 60 with only two portions extending or extendable above the plane of handle face 56: (1) a pin or projection 62 on the distal end of hold-down 60 and (2) a blade retaining bar 64 on the proximal end of the hold-down 60. Interaction between shield 70 and each of blade hold-down arms 642 and projection 62, enables function of the handle assembly 50. Recess 542 in handle 50 may be penetrated by cleaning holes 544 to permit entry of steam or other fluids during disinfecting, cleaning and sterilization.

While numerous other structures and materials may be used for hold-down 60, the embodiment illustrated in the figures may be fabricated from a single piece of precipitation hardenable stainless steel, 400 or 300 series family with two working ends. On its proximal end, hold-down 60 provides a blade retaining bar 64 for contact with the distal blade end 93. Bar 64 is supported in the illustrated embodiment by arms 642 that attach to resistance arms 682 surrounding a hold-down head 63 penetrated by a rivet hole 66. When the components are assembled, rivet hole 66 is positioned on a rivet pin 548 protruding from an up-standing hold-down stand or promontory 546. Stand 546 also includes an optional proximally-facing bracket or centering hold-down limiter 550.

Hold-down 60 also provides a distal end pin or projection 62 formed at an end of the bar 63 structure by a flex arm 628 configured to urge projection 62 upward out of recess 552 and against shield 70 and button 80. A distally-facing tang 622 protruding from the base of projection 62 is received in a recess 590 in handle 50 that defines a lip 592 that overlies tang 622 and thereby limits movement of projection 62 out of recess 552. This recess 590 can be provided by forming a flat-bottom cleaning hole that does not quite penetrate face 56.

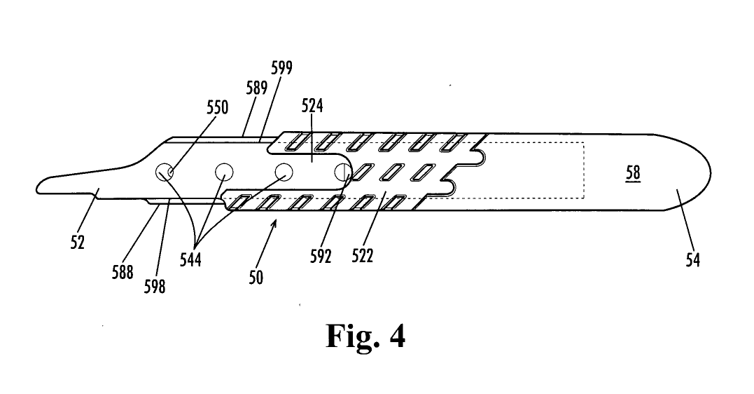

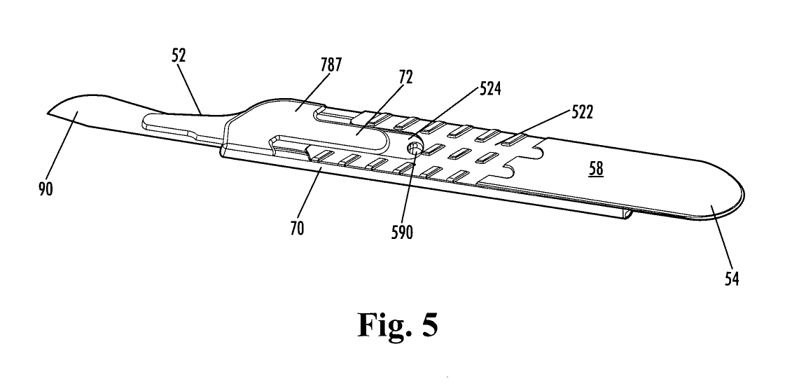

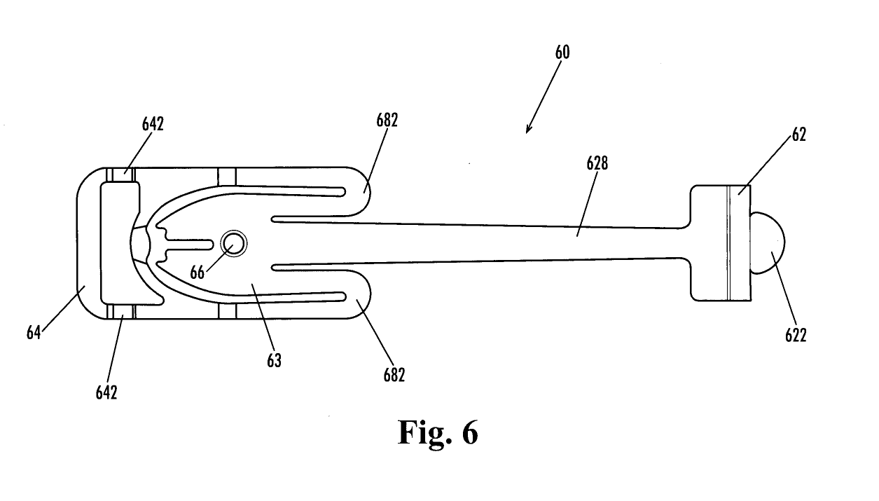

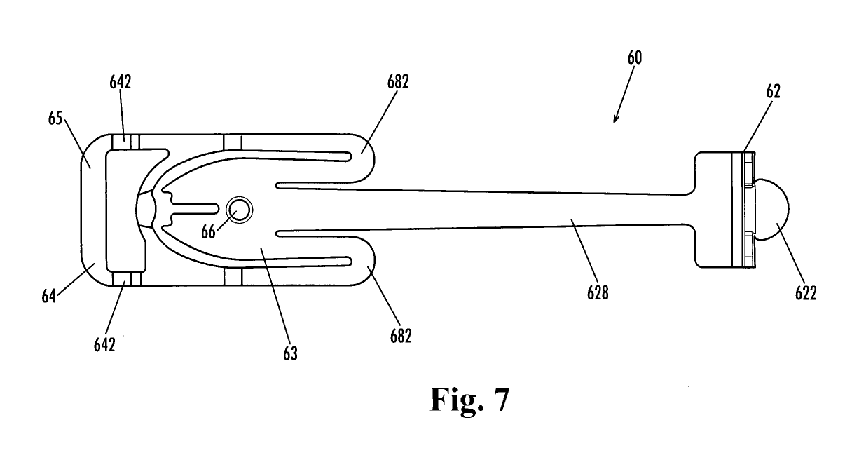

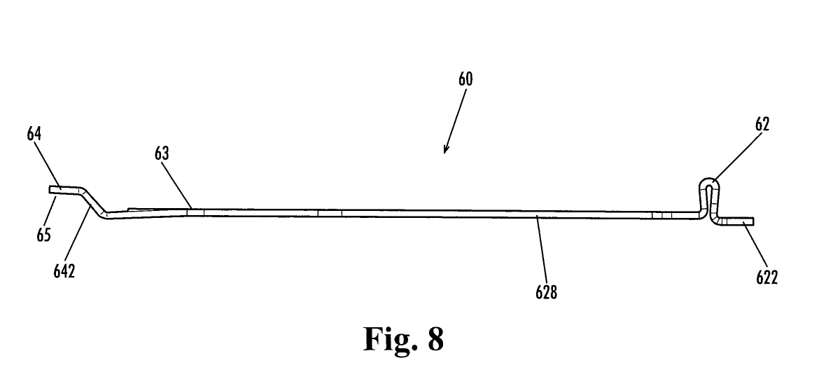

As shown in FIG. 4, the back 58 of the handle 50 includes handle grips 522 extending from the proximal end of the back 58 to nearly the midpoint of the back 58. In the embodiment shown in the figures, the handle grips 522 are a series of raised bumps surrounded by a surface below the bottom face 58, so that the tops of the grips are at least approximately even with the back 58.

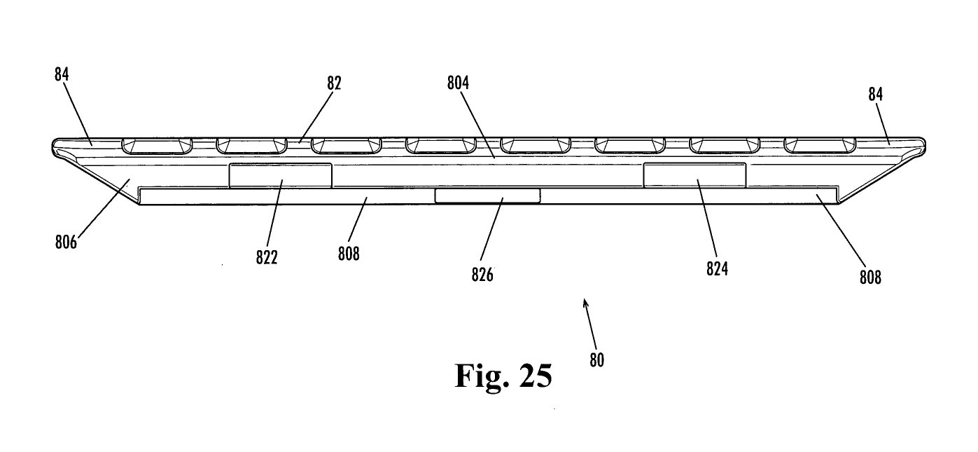

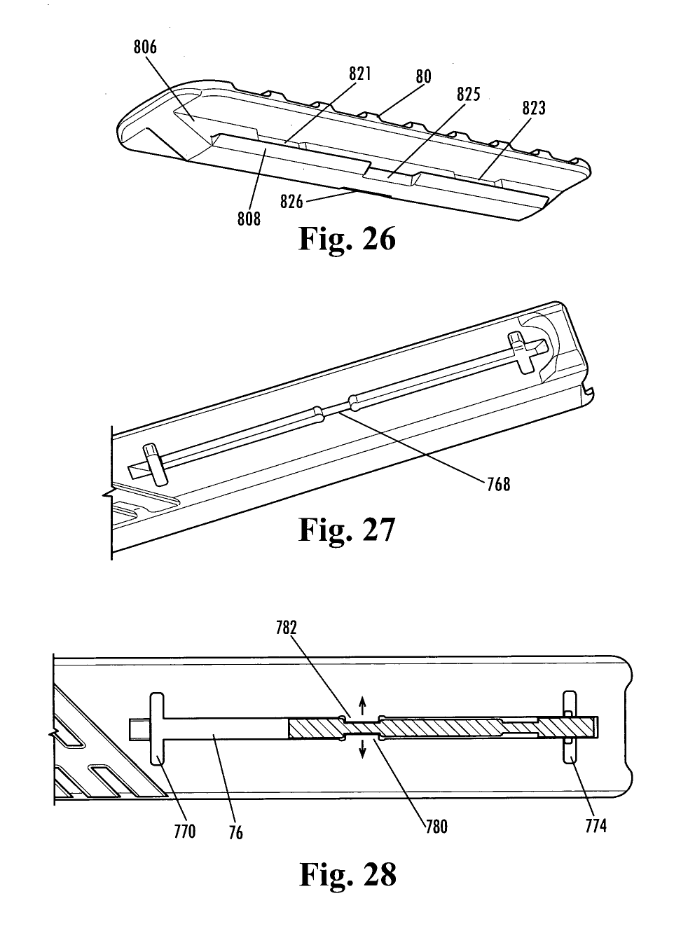

As is illustrated in FIGS. 1, 14-19, and 25-28, an elongated sliding component or button 80 is attached to and slides along a portion of the face 77 of shield 70. Details of one embodiment of the button 80 and portions of the shield 70 are illustrated in FIGS. 26-28. Portions of button 80 have a cross-sectional shape similar to an I-beam where the button face or “top flange” 804 lies against the shield face 77 and is attached by an interconnecting “web” 806 (positioned within a longitudinally-extending slot or travel-way 76 in the shield 70) to a narrower lower face or “bottom flange” 808.

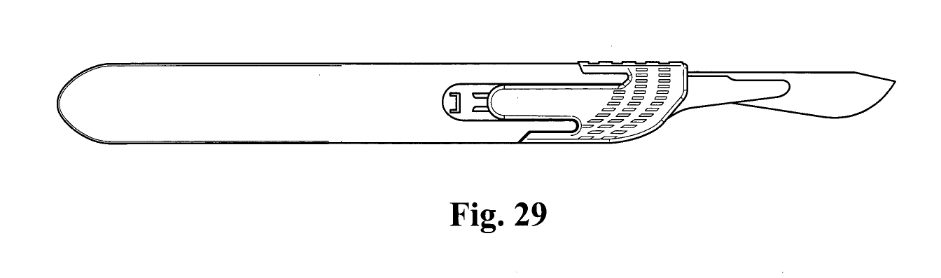

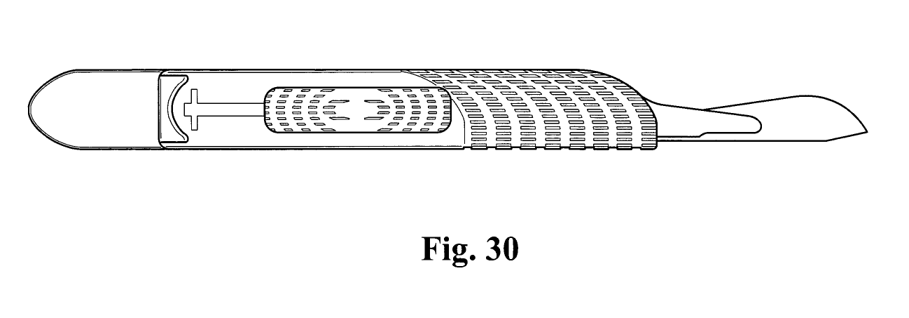

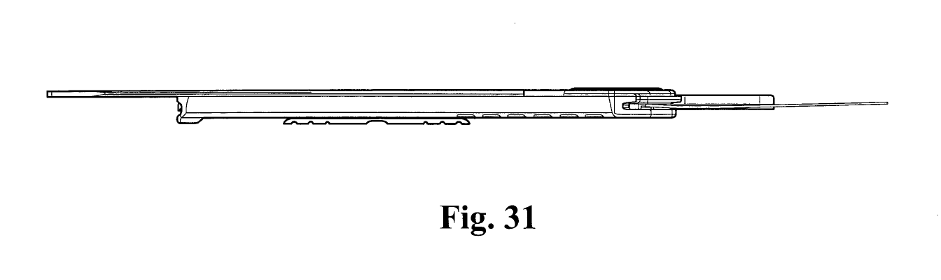

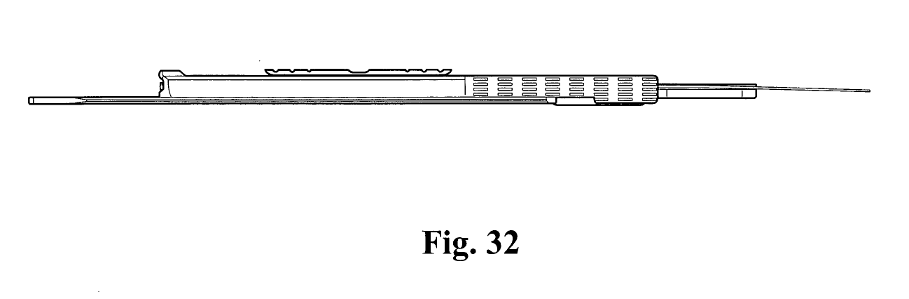

The button 80 includes a plurality of lateral ridges 82. In one embodiment, the lateral ridge on each end of the button is a longer ridge 84. The top surface also includes a long ridge 86 positioned longitudinally. In an alternative embodiment, shown for example in FIGS. 29-32, the ridges are curved, and may include curved arrowhead shapes pointing in the direction of the slide.

The web 806 of the button 80 engages the side walls 784 and 786 of travel-way 76 to lock the button into the travel-way 76. Detent recesses 821, 822, 823, and 824, along the web 806 of button 80 engage protrusions 780 and 782 at either the “blade exposed” or “blade shielded” configuration to reduce the likelihood that button 80 will slip out of position. Cut outs 825 and 826 at the midpoint of bottom plate 808 allow the button to snap into travel-way 76 over protrusions 780 and 782.

Shield 70 is penetrated by transverse detent slots 770 and 774 that can receive projection 62 of locking hold-down 60 when shield 70 is positioned on the handle 50 with one or the other of detent slots 770 or 774 aligned with projection 62 and with button 80 clear of the slot. As either detent slot 770 or 774 aligns with projection 62 the flex arm 628 forces projection 62 upward, creating an audible “click” as projection 62 rises into detent slot 770 or 774, locking the shield 70 in place. Sliding button 80 toward the proximal end of shield 70 covers detent slot 770 pushing projection 62 out of slot 770 (or preventing entry of projection 62 into slot 770 if it is not already in it). Sliding button 80 toward the distal end of shield 70 likewise covers detent slot 774, pushing projection 62 out of, or preventing projection 62 from entering, detent slot 774.

Travel way 76 has a centrally located travel-way stop 768 formed by protrusions 780 and 782 in the side walls 784 and 786 of travel-way 76. The web 806 of the button 80 engages the side walls 784 and 786 to releasably lock the button in desired positions along the travel-way 76. Detent recesses 821, 822, 823, and 824, along the web 806 of button 80 engage protrusions 780 and 782 at either the “blade exposed” or “blade shielded” configuration to prevent the button 80 from slipping out of position. With protrusions 780 and 782 received in recesses 821 and 822, button 80 is held at an extreme of travel within travel-way 76. With protrusions 780 and 782 received in recesses 823 and 824, button 80 is held at the other extreme of travel within travel-way 76. Gaps 825 and 826 in bottom plate 808 of button 80 are aligned over and receive stops 780 and 782 when button 80 is assembled to shield 70.

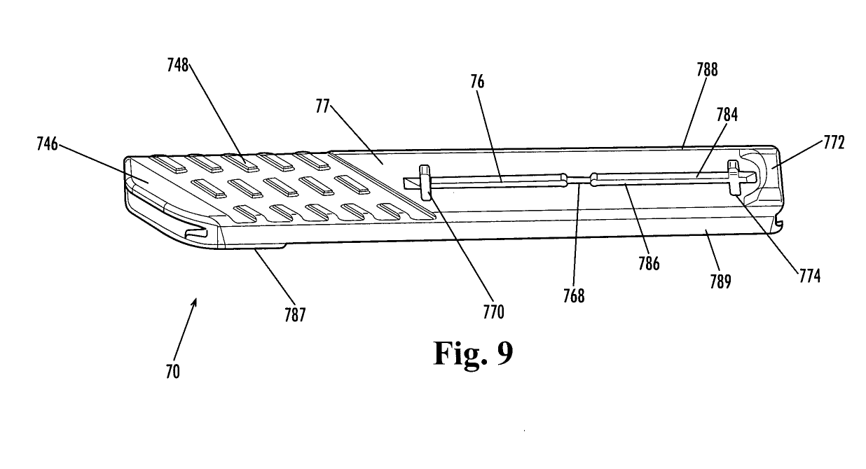

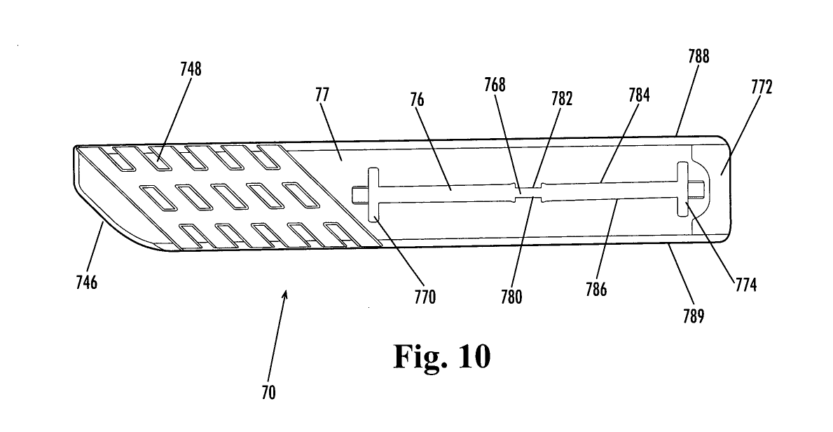

Shield 70 is illustrated in FIGS. 9-11. As noted above, shield 70 generally lies against handle face 56 and has external top and bottom edges 788 and 789 that extend around the top 588 and bottom 589 of handle face 56 and into the grooves 598 and 599 of handle 50, thereby enabling shield 70 to be assembled onto handle 50 and to slide on handle 50 among “blade shielded,” “blade exposed” and “blade removal/replacement” positions.

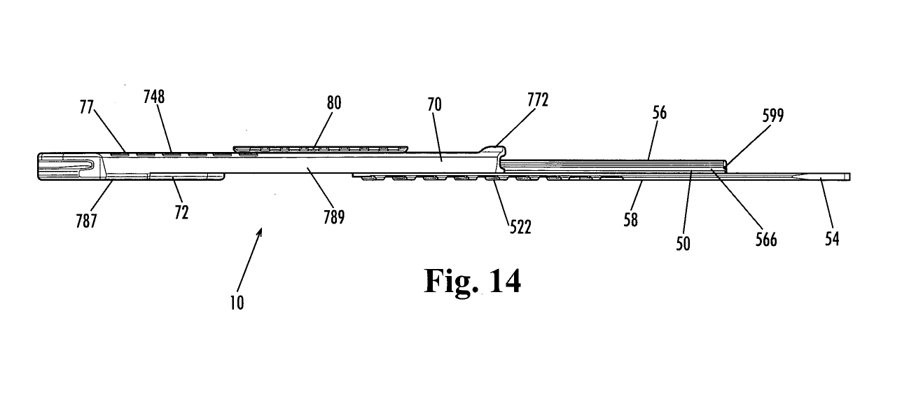

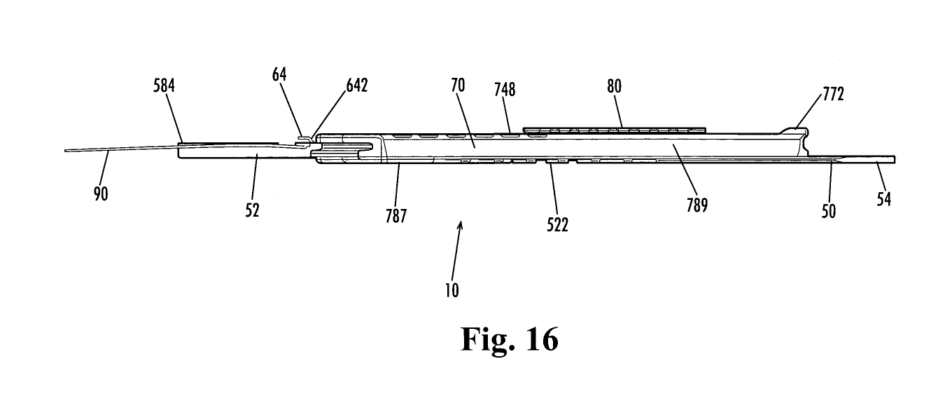

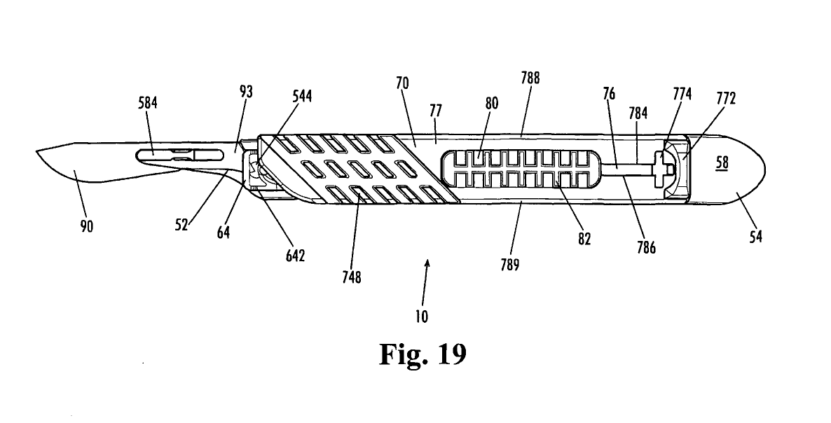

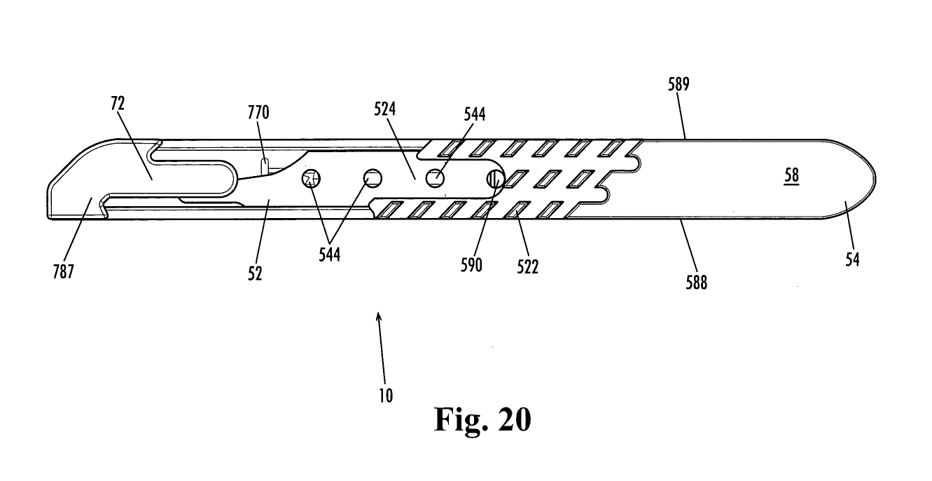

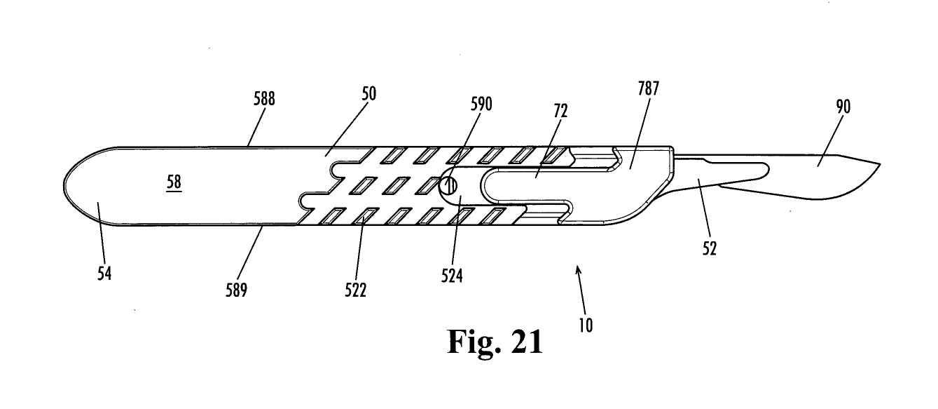

The shield 70 has a generally rectangular but relatively complex cross-sectional shape. A face 77 is equal in width to the width of the handle 50 back 58 and, in the embodiment illustrated in FIGS. 14-16, about two-thirds of the length of the handle 50. Face 77 has projecting “bumps” or grips 748 near its proximal end, and a centrally positioned longitudinal travel-way 76 near its distal end. A bridge 787 connects edges 788 and 789 at the proximal end of shield 70 and supports a shield tongue 72 that extends distally from the bridge 787. This bridge 787 permits shield 70 to entirely encircle handle 50 at the shield 70 proximal end, and bridge 787 and tongue 72 (together with a proximal portion of the face 77 of shield 70) substantially cover blade 90 when shield 70 is in its proximal, “blade shielded” position on handle 50 (as illustrated in FIG. 20). Tongue 72 slides into a tongue recess 524 in handle 50 when shield 70 is in its retracted, blade exposed position, and tongue 72 fully occupies tongue recess 524 when shield 70 is in the blade removal/replacement position illustrated in FIGS. 16 and 19. This configuration provides shield structure on the back of the assembly 10 long enough to substantially cover blade 90 when shield 70 is in the blade covered position while not preventing contact between a user's hand and relatively proximal region of handle 50 in the blade exposed configuration.

The lower, proximal corner 746 of the shield 70 is “eased” or removed to provide unobstructed access to blade 90. The shield grips 748 are defined by removing or omitting adjacent material to establish a surrounding surface below that of shield 70 face 77 around travel-way 76 and the region of face 77 at the proximal end of the shield 70. These grips facilitate sliding shield 70, and lateral ridges 82 on button 80 facilitate application of thumb pressure to button 80 and rise above the shield grips 748, thereby providing a differential tactile response to a user, confirming location of the user's thumb on one or the other of the button 80 or shield 70 grips 748. A distal promontory 772 that rises above shield face 77 to the same plane as lateral ridges 82 provides a tactilely continuous structure when the button abuts the promontory 772. Alternatively, when button 80 is positioned proximally on the shield 70, promontory 772 provides a structure against which thumb pressure can be exerted to slide shield 70 to its most distal, blade-ejecting and -replacement position. In addition, promontory 772 gives added strength to the shield 70 and accommodates inner face 778. The inner face 778 of shield 70 under promontory 772 slopes to facilitate assembly of shield 70 onto handle 50.

Assembly of the exemplary scalpel components of this invention illustrated in the figures is accomplished by positioning button 80 in travel-way 76, positioning locking hold-down 60 in handle 50 recess 542 with rivet pin 548 in rivet hole 66 (compressing hold-down arms 642) and sliding shield 70 onto the proximal end 52 of handle 50 until hold-down projection 62 seats in one of the detent slots 770 or 774 in shield 70.

While holding button 80 in its most proximal position, force is applied distally to shield 70, causing shield 70 to slide as far distally on handle 50 as it is capable of moving. Continued distal force against shield 70 while maintaining a proximal force on button 80 slides shield 70 to the fully extended blade position. This also exposes hold-down 64, allowing hold-down 64 to lift enough to release the distal end 93 of blade 90, allowing it to fall off tang 584. A new blade 90 can then be placed on and held in position on tang 584 while shield 70 is slid proximally on handle 50 until hold down 64 is pressed down by the proximal end of shield 70, and projection 62 is received in detent slot 770 (for use with the blade 90 exposed) or in detent slot 774 (for safe passing with the blade 90 covered).

The blade 90 can be alternatively covered for safe passing or exposed for safe use by application of force, typically with the user's thumb, on the button 80, first, to slide the button 80 within the travel-way 76 and relative to the shield 70 and then to slide the button 80 and shield 70 together until the locking hold-down 60 projection 62 seats in one or the other of the detent slots 770 and 774. Because of the tactile features on the scalpel 10 components and the audible “click” that occurs when the projection 62 seats in a detent slot 770 or 774, a user can very quickly learn to manipulate the shield without looking at the scalpel.

The scalpel assembly of this invention can be produced in numerous alternative embodiments. In other embodiments the hold-down 60 need not be a continuous structure. The projection 62 may be spring loaded, or forced upward by a resistance arm, independent of the hold-down arms 642 and the flex arm 628. The projection 62 also need not travel normal to face 56 and button 80. Other embodiments, for instance may feature a projection that moves parallel to face 56 and/or button 80. The important feature is that the shield 70 locks at two different locations (770 and 774 in the embodiment above).

Another embodiment may locate the blade 90 on the back 58 of the handle, in which case the hold-down arms 642 would have to pull the blade against back 58 or another surface.

Other embodiments may include two stationary buttons instead of one slidable button 80. A separate button at each detent slot 770 and 774 could be able to both receive and disengage projection 62. The user could then apply a force (distally or proximally) and slidably move the shield 70 to a desired position.

Another embodiment may associate projection 62 with detent slot 774 at the blade exposed position, and with detent slot 770 at the blade covered position.

Handle 50 may be machined, molded, forged, cast or otherwise manufactured of steel, stainless steel, high temperature and chemical-resistant plastic such as PEK (poly-ether-ketone), PEEK (poly-ether-ether-ketone) or PEKK (poly-ether-ketone-ketone), or composites of these materials or of any other material using any other technique that results in a handle having acceptable characteristics of strength, rigidity, heft, durability, sterilizability, and cleanability.

Button 80 and shield 70 likewise may be machined, molded, forged, cast or otherwise manufactured of steel, stainless steel, high temperature and chemical-resistant plastic such as PEK (poly-ether-ketone), PEEK (poly-ether-ether-ketone) or PEKK (poly-ether-ketone-ketone) or of any other material using any other technique that results in a handle having acceptable characteristics of strength, rigidity, heft, durability, sterilizability, and cleanability. While not indispensable, it is desirable that one or both of buttons 80 and shield 70, and of shield 70 and handle 50 have properties of lubricity to facilitate sliding contact without the need for additional lubrication.

Locking hold-down 60, or components providing the function of locking hold-down 60, may be fabricated from a similar range of materials utilizing a similar range of techniques provided that such manufacture achieves adequate properties of springiness, resistance, strength, durability, cleanability, sterilizability and the like.

As should be apparent from the foregoing description of this invention and alternative structures, manufacturing techniques, and materials, this invention is not limited by such exemplary embodiments but includes all variations and alternative embodiments within the scope and spirit of the foregoing description, the accompanying drawings, and the following claims.

1. A safety scalpel for use with a replaceable scalpel blade, the scalpel comprising:
a. an elongated handle attached to a blade tang for receiving the blade,
b. a shield positioned to slide on the handle between a first secured position covering the blade and a second secured position exposing a portion of the blade,
c. a button slidably attached to the shield,
d. a blade retainer attached to the handle proximate the tang and movable between:
i. a blade secured position retaining the blade on the tang and
ii. a blade released position permitting the blade to separate from the tang, and
e. a protrusion coupled to the handle and biased to seat in one or the other of two recesses in the shield when the shield is positioned on the handle in either the first, blade-covering position or the second blade-exposing position.

2. The scalpel of claim 1, wherein the shield further comprises a travel way and the button is received in the travel way for slidable attachment to the shield.

3. A method for preparing a scalpel for safe passing after its use, comprising
a. providing a scalpel assembly having:
i. a handle to which a blade is removably attached and a shield is slidably attached and locked in a position with the blade exposed, and
ii. a button slidably secured to the shield,
b. applying proximal pressure to the button to cause the button to slide proximally within the shield to thereby unlock the shield from its position on the handle, and
c. continuing to apply proximal pressure to the button to cause the shield to slide on the handle until it completely covers the blade and locks in position on the handle.

4. A method for preparing a scalpel for use, comprising
a. providing a scalpel assembly:
i. having a handle to which a blade is removably attached and a shield is slidably attached and locked in a position with the blade covered by the shield, and
ii. a button slidably secured to the shield,
b. applying distal pressure to the button to cause the button to slide distally within the shield to thereby unlock the shield from its position on the handle, and
c. continuing to apply distal pressure to the button to cause the shield to slide on the handle until it exposes at least a significant portion of the blade and locks in position on the handle.

5. A safe passing scalpel, comprising:
a. a scalpel assembly:
i. having a handle to which a blade is removably attached and a shield is slidably attached, and
ii. a button slidably secured to the shield, so that
b. applying proximal pressure to the button to cause the button to slide proximally within the shield unlocks the shield from its position on the handle, and
c. continuing to apply proximal pressure to the button causes the shield to slide on the handle until it completely covers the blade and locks in position, after which
d. applying distal pressure to the button to cause the button to slide distally within the shield unlocks the shield from its position on the handle, and
e. continuing to apply distal pressure to the button to cause the shield to slide on the handle until it exposes at least a significant portion of the blade and locks in position on the handle.

6. The safe passing scalpel of claim 5, wherein:
a. with the shield locked in a blade-exposed position on the handle, applying force to retain the button in a proximal position while moving the shield distally unlocks the shield from the handle, and
b. applying distal force on the shield exposes the blade so that it may be released from the handle.
